(19) United States
(12) Patent Application Publication
(10) Pub. No.: US 2010/0256450 A1
(43) Pub. Date: Oct. 7, 2010

(54) LAPAROSCOPE AND SETTING METHOD THEREOF

(76) Inventors: Seung Wook Choi, Gyeonggi-do (KR); Dong Myung Min, Gyeonggi-do (KR)

Correspondence Address: BIRCH STEWART KOLASCH & BIRCH, PO BOX 747, FALLS CHURCH, VA, 22040-0747, US

(21) Appl. No.: 12/747,473

(22) PCT Filed: Jul. 23, 2009
(86) PCT No.: PCT/KR09/04093

§ 371 Date: Jun. 10, 2010

(30) Foreign Application Priority Data
Oct. 24, 2008 (KR) 10-2008-010452224

(51) Int. Cl. A61B 1/06

(52) U.S. Cl. 600/182

(57) ABSTRACT

A laparoscope includes: a housing extending a particular length; a pair of lenses set in both end portions of the housing along a lengthwise direction; a pair of first reflectors installed within the housing adjacent to the pair of lenses to reflect light from the pair of lenses towards a particular position; a second reflector installed within the housing that receives the light reflected from the pair of first reflectors and reflects the light in a particular direction; and an optical channel coupled to the housing that receives the light reflected from the second reflector and transmits the light to a particular position. Since a single-lens laparoscope may be connected to the housing where a pair of lenses are set with a gap in-between, the diameter of the laparoscope can be reduced, and an image can be obtained that has a brightness comparable to that obtained by a single-lens laparoscope.

### CROSS-REFERENCE TO RELATED APPLICATIONS

This application claims foreign priority benefits under 35 U.S.C. .sctn. 119(a)-(d) to PCT/KR2009/004093, filed Jul. 23, 2009, which is hereby incorporated by reference in its entirety.

### BACKGROUND

The present invention relates to a laparoscope and to a method of setting the laparoscope.

In the field of medicine, surgery refers to a procedure in which a medical device is used to make a cut or an incision in or otherwise manipulate a patient's skin, mucosa, or other tissue, to treat a pathological condition. One type of surgery, a laparotomy, is a surgical procedure in which the skin of the abdomen is cut open and an internal organ, etc., is treated, reconstructed, or excised.

When conducting a laparotomy, an incision is made in the skin and a particular amount of space is formed between the skin and the tissue, with the surgical operation performed within this space. As this may increase scars and prolong the healing period, laparoscopic surgery has recently been proposed as an alternative.

In laparoscopic surgery, a small incision is made in the surgical site of the patient, through which a laparoscope is inserted, so that the surgery may be conducted while observing the surgical site within the abdominal cavity. Laparoscopic surgery is widely used in various fields of medicine, including internal medicine, surgery, urology, gynecology, and obstetrics. The laparoscope is an apparatus used for the imaging diagnosis of an internal organ and typically involves an apparatus installed with a miniature camera that is inserted in the body, so that the image information retrieved by the miniature camera may be observed through an external monitor.

The conventional laparoscope can be divided into the single-lens type, which includes one lens set in an end portion of a cylindrical tube, and the stereo type, which includes a pair of lenses. The single-lens type provides a relatively brighter image, but is able to provide only a planar image that does not render a 3-dimensional look, whereas the stereo type is able to provide a 3-dimensional image, but provides an image that is not as bright. In addition, even when a stereo laparoscope is used, the 3-dimensional effect of the image may not be significant, as the pair of lenses may be set within a range of about 12 mm, the diameter of a typical laparoscope.

In “robot surgery,” which is performed using a laparoscope and a surgical robot inserted in the patient's body, an incision may be made in the patient's skin, and a trocar may be inserted that serves as a port through which the laparoscope and surgical instruments are inserted. Since the laparoscope may require a minimum cross section for setting a pair of lenses as described above, it is unsuitable to indiscriminately reduce the size of the laparoscope, unlike other surgical equipment. Consequently, in the case of robot surgery, the trocar used for inserting the laparoscope currently has the largest diameter.

In conventional laparoscopic surgery, a gas such as CO2 may be injected into the portion photographed with the laparoscope, in order to obtain a space required for photography. The injected gas is not harmless to the human body, and there is also a risk of a medical accident if the gas pressure is not properly regulated.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a laparoscope and a method of setting the laparoscope, which can be used to obtain an image having a significantly 3-dimensional effect and having a brightness comparable to that of the single-lens type, even with a reduced diameter of the laparoscope, which do not require the injection of a gas such as CO2 to obtain a space for laparoscopic photography, and which do not require making an incision in the skin for inserting the laparoscope.

One aspect of the present invention provides a laparoscope that includes: a housing, which extends a particular length; a pair of lenses, which are set in both end portions of the housing along a lengthwise direction; a pair of first reflectors, which are installed within the housing adjacent to the pair of lenses to reflect light from the pair of lenses towards a particular position; a second reflector, which is installed within the housing, and which receives the light reflected from the pair of first reflectors and reflects the light in a particular direction; and an optical channel, which is coupled to the housing, and which receives the light reflected from the second reflector and transmits the light to a particular position.

The second reflector can undergo oscillatory rotation at a particular frequency, to receive the light reflected from the pair of first reflectors and reflect the light respectively towards the optical channel. Here, the oscillatory rotation frequency of the second reflector can be 60 to 120 Hz.

An optical receiver that includes a CCD can be coupled to an end portion of the optical channel. The optical channel can be detachably coupled to the housing. For example, a magnet can be attached to one or more of the optical channel and the housing, such that the optical channel is coupled to the housing by magnetic force. In another example, the optical channel can have an alignment protrusion formed on the end portion coupled to the housing, while the housing can have an indentation, which mates with the alignment protrusion, formed in the portion coupled to the optical channel

The optical channel can be coupled to the housing along a same lengthwise direction as that of the housing. Alternatively, the housing can be hinge-coupled to the optical channel, such that the housing is rotatable between an orientation having a same lengthwise direction as that of the optical channel and an orientation orthogonal to the optical channel

Another aspect of the present invention provides a laparoscope that includes: a housing, which extends a particular length; a pair of lenses, which are set in both end portions of the housing along a lengthwise direction; an optical receiver, which is installed within the housing adjacent to the pair of lenses to receive light from the pair of lenses respectively and convert the light into electrical signals; and an electrical contact, which is electrically connected to the optical receiver and exposed at an exterior of the housing.

The housing can have a cylindrical shape with a diameter large enough for a trocar inserted in the surgical site to pass through. The pair of lenses can be set in the housing facing the same direction. The laparoscope can further include a lighting part coupled to the housing to irradiate light towards the direction in which the pair of lenses are facing.

Also, the laparoscope can further include a support, which is detachably coupled to the housing, and which extends along a particular lengthwise direction. Here, the support can be coupled to the housing along a same lengthwise direction as the housing, or the housing can be hinge-coupled to the support, such that the housing is rotatable between an orientation having a same lengthwise direction as that of the support and an orientation orthogonal to the support.

A needle coupled to the housing can additionally be included, in which case the needle can be shaped as a hollow tube, through which the electrical contact may be exposed to the exterior, or the electrical contact can be formed at an end portion of the needle. The needle can be hinge-coupled to the housing, such that the needle is rotatable between an orientation having a same lengthwise direction as that of the housing and an orientation orthogonal to the housing.

Yet another aspect of the present invention provides a method of setting a laparoscope that includes: inserting a laparoscope module, to which a needle is hinge-coupled, into a measurement site; rotating the needle and protruding the needle to an outside of the measurement site; providing a measurement space around the laparoscope module by pulling the needle; and activating the laparoscope.

Additional aspects, features, and advantages, other than those described above, will be obvious from the claims and written description below.

According to a preferred embodiment of the present invention, a single-lens laparoscope may be connected to a housing in which a pair of lenses are set with a particular gap in-between, so that the diameter of the laparoscope can be reduced, and an image can be obtained that has a brightness comparable to that obtained by a single-lens laparoscope. Since the pair of lenses can be separated as necessary to a distance similar to the distance between human eyes, an image can be obtained that provides a 3-dimensional effect similar to that observed by the naked eye. Furthermore, by mounting a lighting device, such as LED's, etc., onto the housing, the laparoscopic surgery can be performed with a much wider range of vision.

Also, the housing equipped with the lenses and a CCD can be inserted as a laparoscope module into the body, after which a space can be obtained by pulling on the laparoscope module. In this way, the space required for laparoscopic photography can be obtained without injecting a gas, such as CO2, etc. The laparoscope module can be inserted through an incision made for a different purpose, and only the needle can be protruded outside. Thus, since there is no need to make an incision in the skin for inserting the laparoscope, a safer form of “minimally invasive surgery” can be implemented.

### BRIEF DESCRIPTION OF THE DRAWINGS

FIG. 1 is a cross-sectional view of a laparoscope according to a first preferred embodiment of the present invention.

FIG. 2 is a cross-sectional view of a laparoscope according to a second preferred embodiment of the present invention.

FIG. 3 is a plan view of a laparoscope according to a third preferred embodiment of the present invention.

FIG. 4 is a plan view of a laparoscope according to a fourth preferred embodiment of the present invention.

FIG. 5 is a cross-sectional view of a laparoscope according to a fifth preferred embodiment of the present invention.

FIG. 6 is a plan view of a laparoscope according to a sixth preferred embodiment of the present invention.

FIG. 7 is a plan view of a laparoscope according to a seventh preferred embodiment of the present invention.

FIG. 8 is a flowchart illustrating a method of setting a laparoscope according to a preferred embodiment of the present invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

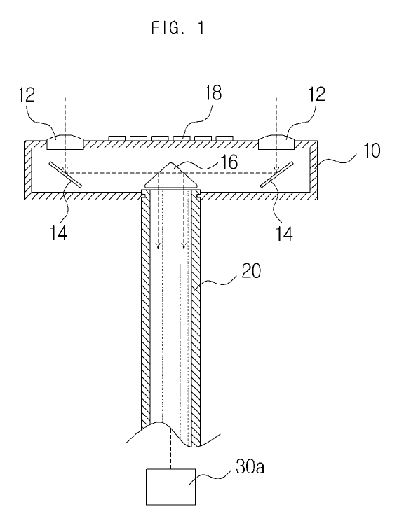

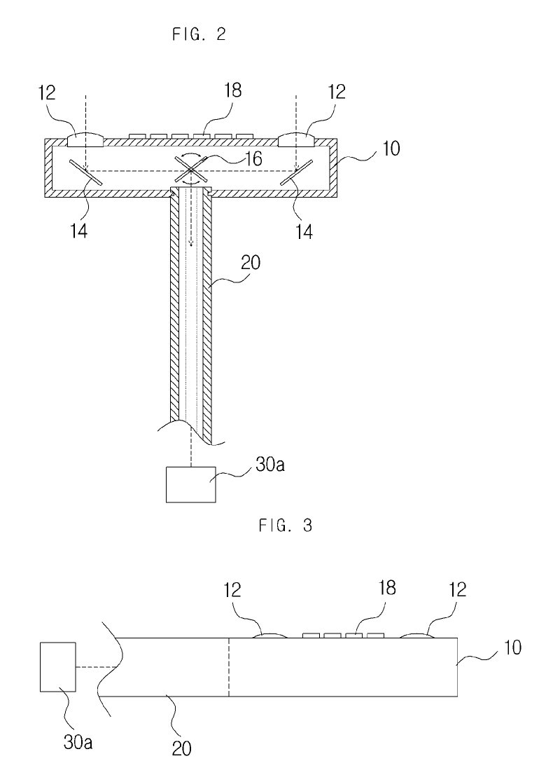

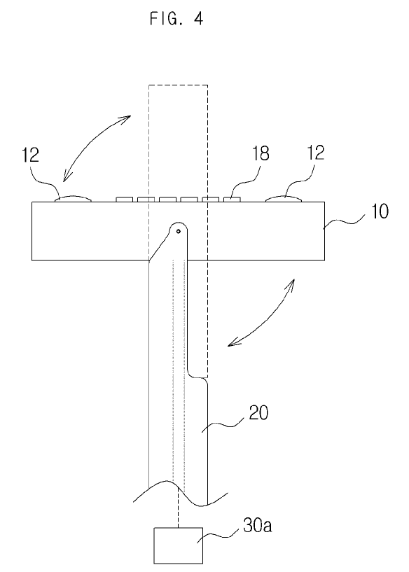

FIG. 1 is a cross-sectional view of a laparoscope according to a first preferred embodiment of the present invention, FIG. 2 is a cross-sectional view of a laparoscope according to a second preferred embodiment of the present invention, FIG. 3 is a plan view of a laparoscope according to a third preferred embodiment of the present invention, and FIG. 4 is a plan view of a laparoscope according to a fourth preferred embodiment of the present invention. Illustrated in FIG. 1 to FIG. 4 are a housing 10, lenses 12, first reflectors 14, a second reflector 16, a lighting part 18, an optical channel 20, and an optical receiver 30a.

This embodiment discloses a laparoscope which resolves the drawbacks of conventional stereo laparoscopes, namely, the limitation in reducing the diameter and the low brightness of the image, and which provides a superb 3-dimensional image comparable to that observed by the naked eye.

A conventional stereo laparoscope may include a pair of lenses at one end of the laparoscope, to obtain an image from each of the lenses and thus form a visual image that provides a 3-dimensional effect. Using the principle of binocular disparity, where the difference in position of an object as seen by either eye of a person generally depends on the distance of the object, the conventional stereo laparoscope can provide a seemingly 3-dimensional image.

However, since the conventional stereo laparoscope requires the setting of a pair of lenses, there is a limit to how much the diameter of the laparoscope can be reduced, and since there is not a sufficient distance between the pair of lenses, the 3-dimensional effect may not be significant.

For example, if a pair of lenses each having a diameter of 5 mm are set into the end portion of a laparoscope having a diameter of 12 mm, the distance between the lenses may be about 6 mm, so that the image obtained by the pair of lenses may not provide a significantly 3-dimensional effect. Also, having to include two lenses, it may not be possible to reduce the diameter of the laparoscope any further.

In the present embodiment, a pair of lenses can be set with the distance in-between similar to the distance between the eyes of an actual person, so that the 3-dimensional effect may be greatly improved compared to that of the conventional stereo laparoscope.

That is, the structure of a laparoscope according to the present embodiment can include the pair of lenses 12 set in the housing 10 with a particular distance in-between, where the light from the lenses 12 may be reflected and then transmitted via the optical channel 20 to the optical receiver 30a. The optical channel 20 can be modified from an existing laparoscope such that the portion coupled to the housing 10 may be detachably joined to the housing 10.

If a mirror undergoing oscillatory rotation at a particular frequency is used to transmit the light from each of the pair of lenses 12 respectively to the optical channel 20, as will be described later in more detail, an existing single-lens laparoscope can be applied unaltered as the optical channel 20. As such, the diameter of the laparoscope, i.e. the diameter of the optical channel 20 can be greatly reduced, compared to that of the conventional stereo laparoscope.

The housing 10 may be shaped as a tube that extends along a particular length, such that the pair of lenses 12 may be arranged with a particular gap in-between. In the example shown in FIG. 1, the housing 10 is formed as a cylindrical pipe, and the pair of lenses 12 are set in both ends of the housing 10, so that the pair of lenses 12 are separated from each other by the length of the housing 10. Therefore, in a laparoscope according to the present embodiment, the gap between the pair of lenses 12 may be increased simply by increasing the length of the housing 10 correspondingly.

A reflector may be installed within the housing 10 to transfer the light from the lenses 12 to the optical channel 20. That is, a pair of first reflectors 14 may be installed adjacent to the pair of lenses 12, to reflect the light from the lenses 12 respectively to a second reflector 16, and the second reflector 16 may be installed to receive the light from the pair of first reflectors 14 and reflect the light to the optical channel 20. Various optical systems, such as mirrors and prisms, etc., which are capable of altering an optical path, can be applied as the first reflectors 14 and second reflector 16.

FIG. 1 illustrates an mechanism in which mirrors are set as the first reflectors 14 and a prism is set as the second reflector 16 to transfer the light from the lenses 12 to the optical channel 20, but the present invention is not thus limited, and it is obvious that various other optical systems for transferring the light from the lenses 12 to the optical channel 20 can be used in addition to or in place of the first and second reflectors 14, 16.

In this way, the light from the lenses 12 may be transferred by the first and second reflectors 14, 16 to the optical channel 20, and may then be transmitted via the optical channel 20 to an optical receiver 30a coupled to the end of the optical channel 20. The optical receiver 30a may be equipped with a device for receiving and processing image information, such as a CCD (charge-coupled device), etc., so that the image photographed by the laparoscope may be displayed on a monitor.

As already described above, the laparoscope according to the present embodiment can utilize a typical single-lens laparoscope as the optical channel 20, to obtain and process a 3-dimensional image, so that the diameter of the laparoscope may greatly be reduced. For the laparoscope according to the present embodiment, it is also possible to insert the housing 10 portion and the optical channel 20 separately into the body, and then join the housing 10 to the optical channel 20 inside the body for use. The action of joining the housing 10 to the optical channel 20 can be performed manually, and in the case of robot surgery, the joining can be performed by manipulating a robot arm inserted into the body for surgery.

Thus, the housing 10 according to the present embodiment can have a cylindrical shape, so that the housing 10 may be inserted separately into the body. The diameter of the housing 10 may be such that allows the housing 10 to pass through the trocar, which is inserted in the surgical site of the patient to facilitate the insertion of a laparoscope or a robot arm. A surgical trocar is a tube-shaped medical tool, typically used for accessing the abdominal cavity. The trocar may be inserted in the surgical site, and may then serve as a passageway through which a laparoscope or a medical tool, such as surgical instruments, etc., may be inserted.

Thus, according to the present embodiment, there is no need to make an additional incision in the surgical site for inserting the laparoscope into the body. The housing 10 and the optical channel 20 may be inserted separately through a previously made incision for inserting the laparoscope, etc., i.e. the portion where the trocar is inserted, after which the housing 10 and the optical channel 20 may be joined together for use.

As illustrated in FIG. 1, the pair of lenses 12 set in the housing 10 can be set in the same direction, i.e. the direction in which photography is desired. In order to obtain a clearer and more detailed image, a lighting part 18 may additionally be set, for example by setting a number of LED's between the pair of lenses 12, to provide a sufficient amount of light to the portion being photographed. However, the lighting part 18 according to the present embodiment does not necessarily have to be set between the pair of lenses 12, and it is obvious that the lighting part 18 can be set in a variety of positions and directions that enables the lighting part 18 to irradiate a sufficient amount of light to the portion photographed by the lenses 12.

As the light from the pair of lenses 12 are reflected by the first reflectors 14, respectively, and transferred to the second reflector 16, the second reflector 16 may receive a pair of image information sets. In order to reflect the pair of image information sets to the optical channel 20, the second reflector 16 can be formed in the shape of a prism, as in the example shown in FIG. 1. In this case, an existing stereo laparoscope can be used for the optical channel 20, so that the pair of image information sets reflected by the second reflector 16 may be transmitted simultaneously to the optical receiver 30a.

Alternatively, when using a single-lens laparoscope for the optical channel 20 as described above to reduce the diameter of the laparoscope, the second reflector 16 can be formed as a mirror that undergoes oscillatory rotation at a particular frequency, as in the example shown in FIG. 2. In other words, the pair of image information sets entering the second reflector 16 may be reflected alternately to the optical channel 20.

For example, if an image information set from one lens 12 is to be received at 30 fps (frames per second), the second reflector 16 can be oscillated at 60 Hz, so that the image information obtained from one of the lenses 12 may be transmitted at 30 frames per second to the optical receiver 30a. Likewise, if an image of 60 fps is desired, the second reflector 16 may be rotated at 120 Hz.

The image signals transferred to the optical channel 20 via the lenses 12 and the first and second reflectors 14, 16 may be transmitted through the optical channel 20. The optical receiver 30a, which includes a CCD, etc., may be coupled to the end of the optical channel 20, to convert the image signals into electrical signals. The image signals converted into electrical signals can be displayed on a monitor, allowing the surgeon to conduct surgery while viewing the image of the abdominal cavity on a screen.

By obtaining the image information of the inside of the abdominal cavity through a pair of lenses 12 that are separated by a sufficient distance, the surgeon can view the image with a 3-dimensional effect similar to that provided when a person looks inside the abdominal cavity with the naked eye. For example, by setting the pair of lenses 12 with a gap of about 6 cm, similar to the distance between a person's eyes, a 3-dimensional image can be obtained that is substantially the same as that seen by the naked eye.

Furthermore, by setting a lighting part 18 in the housing 10, as described above, to provide a sufficient amount of light, an image can be obtained for a far greater area than that which was possible for a conventional laparoscope, whereby the accuracy and stability of the laparoscopic surgery may be radically improved.

Since the optical channel 20 according to the present embodiment may be inserted in the abdominal cavity independently of the housing 10 and may be coupled to the housing 10 afterwards, the optical channel 20 can be detachably coupled to the housing 10. When using an existing laparoscope for the optical channel 20 as described above, the end portion of the laparoscope can be modified to a structure that enables joining to the housing 10.

This can be achieved by forming an alignment protrusion (not shown) on an end portion of the optical channel 20 and forming a corresponding indentation (not shown) in the housing 10, so that the optical channel 20 and the housing 10 may be automatically aligned simply by mating the alignment protrusion with the indentation when coupling the optical channel 20 with the housing 10. Of course, it is also possible to form the alignment indentation in an end portion of the optical channel 20 and form a corresponding protrusion that mates with the alignment indentation on the housing 10, and it is obvious that various other mechanical structures may be utilized to align the optical channel 20 with the housing 10.

In addition, it is possible to attach a pair of magnets (not shown) respectively to opposing portions of the optical channel 20 and the housing 10, so that the optical channel 20 and the housing 10 may be attached and detached by magnetic force. The pair of magnets can be a pair of permanent magnets or electromagnets that applies an attractive force on each other, or a mixed set of a permanent magnet and an electromagnet. It is also possible to attach a magnet to one of the optical channel 20 and the housing 10 and attach a magnetic substance, such as a piece of metal, to the other.

As set forth above, the laparoscope according to the present embodiment is a system that uses a pair of lenses 12 to collect image information and uses an optical channel 20 and an optical receiver 30a to process the information into a binocular image. That is, through the shared use of the optical channel 20, as well as the CCD camera coupled to the end of the optical channel 20, a binocular image can be obtained using only a single optical receiver 30a.

As illustrated in FIG. 3, the optical channel 20 does not necessarily have to be coupled to the housing 10 orthogonally, and the optical channel 20 can be coupled to the housing 10 such that the lengthwise direction of the optical channel 20 matches the lengthwise direction of the housing 10. In this case, the housing 10 and the optical channel 20 of the laparoscope according to the present embodiment do not have to be joined after being inserted separately into the body, and may be inserted into the body through the trocar, with the housing 10 already joined to the optical channel 20.

Also, in the embodiment illustrated in FIG. 3, the optical channel 20 and the housing 10 do not necessarily have to be formed as a detachable structure, and may be used as an integrated part.

As illustrated in FIG. 4, the housing 10 can be hinge-coupled to the optical channel 20, such that the housing 10 is able to rotate in relation to the optical channel 20. When the laparoscope according to the present embodiment is inserted into the body, the housing 10 can be positioned to have the same lengthwise direction as that of the optical channel 20, and after the laparoscope is inserted into the body, the housing 10 can be rotated such that the housing 10 is orthogonal to the optical channel 20, to continue with the laparoscopic photography.

In this case also, it is not necessary to join the housing 10 and the optical channel 20 after inserting the housing 10 and the optical channel 20 separately into the body. The housing 10 can be inserted into the body through the trocar while joined to the optical channel 20, and afterwards the housing 10 can be rotated for use.

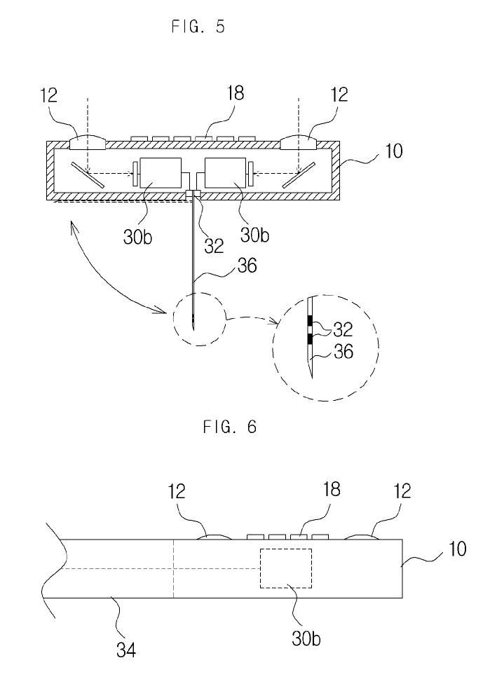

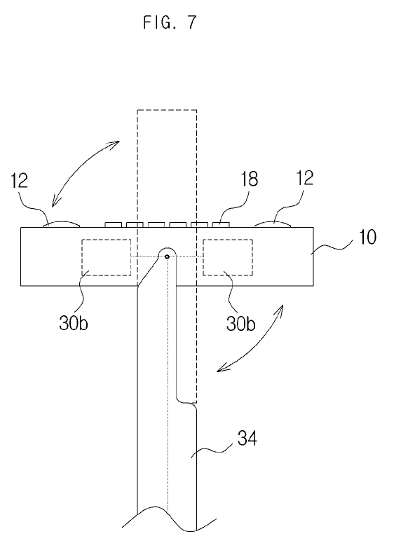

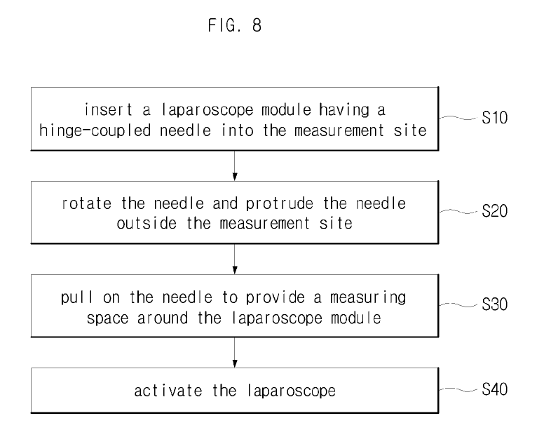

FIG. 5 is a cross-sectional view of a laparoscope according to a fifth preferred embodiment of the present invention, FIG. 6 is a plan view of a laparoscope according to a sixth preferred embodiment of the present invention, FIG. 7 is a plan view of a laparoscope according to a seventh preferred embodiment of the present invention. Illustrated in FIG. 5 to FIG. 7 are a housing 10, lenses 12, a lighting part 18, optical receivers 30b, an electrical contact 32, a support 34, and a needle 36.

In this embodiment, the optical receivers 30b, such as CCD's, etc., may be built in directly inside the housing 10, so that the optical channel 20 of the previously described embodiment may be omitted. That is, a laparoscope according to the present embodiment can include a pair of lenses 12, which are set in the housing 10 with a particular distance in-between, and optical receivers 30b, which receive image information from the lenses 12 and, without relaying the information to an optical channel 20, convert the information directly into electrical signals for transmission.

In this case, the image information converted into electrical signals can be transmitted by way of wires, etc., instead of the optical channel 20, so that the diameter of the laparoscope may be radically reduced to a level incomparable to conventional laparoscopes.

The housing 10 may be shaped as a tube that extends along a particular length, such that the pair of lenses 12 may be arranged with a particular gap in-between, and by setting the pair of lenses 12 in both ends of the housing 10, the pair of lenses 12 can be separated from each other by the length of the housing 10, similar to the previously described embodiment.

In the present embodiment, the optical receivers 30b may be installed within the housing 10 adjacent to the lenses 12, so that the light from the lenses 12 may be received directly by the optical receivers 30b or may be altered by mirrors and then received by the optical receivers 30b. The optical receivers 30b can include a pair of CCD's corresponding with the lenses 12, respectively, to receive the light from the pair of lenses 12 and convert the light into electrical signals.

While FIG. 5 illustrates an example in which the light from the lenses 12 is reflected by mirrors to be received by the optical receivers 30b, the present invention is not thus limited. It is obvious that the optical receivers 30b can be set without mirrors directly behind the lenses 12 and that various other optical systems can be used for transferring the light from the lenses 12 to the optical receivers 30b.

In this way, the light from the lenses 12 can be transferred to the optical receivers 30b and converted into electrical signals, which in turn can be transmitted to the exterior through an electrical contact 32 connected to the optical receiver 30b, so that the image photographed by the laparoscope may be displayed on a monitor. Thus, the image signals converted into electrical signals can be displayed on a monitor, allowing the surgeon to conduct surgery while viewing the image of the abdominal cavity on a screen.

With the laparoscope according to the present embodiment, the image information converted into electrical signals can be transmitted and processed using wires, etc., without using the optical channel 20, so that the diameter of the laparoscope can be radically reduced. Thus, laparoscopic photography can be performed after inserting the housing 10 portion of a laparoscope according to the present embodiment, and connecting a wire to the electrical contact 32 exposed at the exterior of the housing 10.

Similar to the previously described embodiment, the housing 10 according to the present embodiment can have a cylindrical shape, with a diameter that enables the housing 10 to pass through the trocar, so that the housing 10 may readily be inserted into the body.

As illustrated in FIG. 5, the pair of lenses 12 set in the housing 10 can be set in the same direction, i.e. the direction in which photography is desired. Similar to the previously described embodiment, a lighting part 18 may additionally be set, for example by setting a number of LED's between the pair of lenses 12, to provide a sufficient amount of light to the portion being photographed, so that a clearer and more detailed image may be obtained.

As already described above, by obtaining the image information of the inside of the abdominal cavity through a pair of lenses 12 that are separated by a sufficient distance, the surgeon can view the image with a 3-dimensional effect similar to that provided when a person looks inside the abdominal cavity with the naked eye, and by setting a lighting part 18 in the housing 10 to provide a sufficient amount of light, an image can be obtained for a far greater area than that which was possible for a conventional laparoscope, whereby the accuracy and stability of the laparoscopic surgery may be radically improved.

The laparoscope according to the present embodiment may be used by inserting the housing 10 into the body, and then electrically connecting the electrical contact 32 with an external device. The connection between the electrical contact 32 and the exterior can be implemented, for example, by a method of inserting the housing 10 with the wire connected beforehand to the electrical contact 32 and by a method of inserting the housing 10 and the wire into the body separately and then connecting the wire to the electrical contact 32 either manually or by manipulating a robot arm.

Also, as will be described later in more detail, another method can include inserting the housing 10 into the body with a needle-shaped support coupled to the housing 10, protruding the needle 36 out of the body, and connecting a wire, etc., to an electrical contact 32 formed on the needle 36.

Thus, according to the present embodiment, there is no need to make an additional incision in the surgical site for inserting the laparoscope into the body. The housing 10 can be inserted through an incision made for a different purpose, i.e. the portion where the trocar is inserted, after which the electrical contact 32 may simply be connected to the exterior for use.

According to the present embodiment, the housing 10 can be coupled with a support 34 that extends along a particular lengthwise direction, as illustrated in FIG. 6 or FIG. 7, instead of the optical channel 20 of the previously described embodiment. The support 34 may serve to provide mechanical support when the housing 10 is inserted into the abdominal cavity and may be detachably coupled with the housing 10.

A built-in wire in the support 34 can be electrically connected with the optical receivers 30b installed in the housing 10. Then, the housing 10 may be inserted into the body while coupled to the support 34, and the electrical contact exposed at the other end of the support 34 may be connected with an external device.

As illustrated in FIG. 6, the support 34 can be coupled to the housing 10 such that the lengthwise direction of the support 34 matches the lengthwise direction of the housing 10. In this case, the laparoscope according to the present embodiment provides the advantage that the housing 10 can be inserted through the trocar and into the body directly while coupled to the support 34. Also, in the example illustrated in FIG. 6, the support 34 and the housing 10 do not necessarily have to be formed as a detachable structure, and may be used as an integrated part.

As illustrated in FIG. 7, the housing 10 can be hinge-coupled to the support 34, such that the housing 10 is able to rotate in relation to the support 34. When the laparoscope according to the present embodiment is inserted into the body, the housing 10 can be positioned to have the same lengthwise direction as that of the support 34, and after the laparoscope is inserted into the body, the housing 10 can be rotated such that the housing 10 is orthogonal to the support 34, to continue with the laparoscopic photography. In this case also, the housing 10 can be inserted into the body through the trocar while joined to the support 34, and afterwards the housing 10 can be rotated for use.

As illustrated in FIG. 5, a needle 36 can be coupled to a housing 10 according to the present embodiment, and after the housing 10 is inserted into the body, the needle 36 can be protruded to the outside of the body, so that a wire, etc., may be connected to the electrical contact 32 formed on the needle 36.

That is, when a needle 36 is coupled to the housing 10, as illustrated in FIG. 5, the needle 36 can be made to serve as the support 34 and/or the electrical contact 32, by protruding the needle 36 out of the body after the housing 10 is inserted into the body.

A needle 36 having a diameter of about 2 mm, such as a syringe needle, for example, does not leave a scar when it penetrates the skin. Thus, when a needle 36 is coupled to the housing 10 and used as a support, as in the present embodiment, there is no need to make an incision in the patient's skin for inserting the laparoscope. Since the trocar for inserting the laparoscope has the largest diameter, omitting the procedure of making an incision for inserting the laparoscope means that the surgery may be performed with a minimal amount of incisions in the skin

If the needle 36 according to the present embodiment is formed as a hollow tube, as in the case of a syringe needle, the electrical contact 32 can be protruded outwards through the needle 36, so that the electrical contact may be exposed to the exterior simply by protruding the needle 36 out of the body.

Furthermore, by forming the electrical contact 32 at an end portion of the needle 36, as illustrated in FIG. 5, the laparoscope can be electrically connected with an external device by connecting a wire to the needle 36 protruding out of the body. By connecting the electrical contact 32 exposed through the needle 36 to an external device, the image information obtained within the abdominal cavity and converted into electrical signals can be displayed on an external monitor, etc.

Related to this, instead of having the housing 10 coupled to the needle 36, it is also possible to penetrate a needle, on which an electrical contact is formed, into the body from the outside and connect the needle with the electrical contact 32 of the housing 10, so that the laparoscope may be electrically connected with the external device. Obviously, in this case, the electrical contact 32 formed on the housing 10 and the electrical contact formed on the end portion of the needle may be formed to correspond with each other.

For the purpose of inserting the housing 10 into the body with a needle 36 coupled to the housing 10 and then protruding the needle 36 from inside the body to the outside, the needle 36 can be hinge-coupled to the housing 10. Thus, when the housing 10 is inserted into the body, the needle 36 can be folded, to have the same lengthwise direction as that of the housing 10, and after the housing 10 is inserted, the needle 36 can be unfolded, to be orthogonal to the housing 10, so that the needle 36 may penetrate the skin and protrude out of the body.

When performing laparoscopic photography using a housing 10 that has a needle 36 coupled on in this manner, the space required for laparoscopic photography may readily be obtained, without having to inject a gas such as CO2, etc., for expanding the abdominal cavity as in the related art.

That is, with the housing 10 inserted into the body and the needle 36 protruded out of the body, by pulling the needle 36 with an external device, the skin may be pulled around the portion where the housing 10 is inserted, providing a space in the abdominal cavity. Thus, the skin can be pulled just by pulling on the laparoscope, without having to inject a gas such as CO2, etc., which is not harmless to the human body, and in this way, the space required for laparoscopic photography and surgery can readily be obtained.

As such, it may be advantageous to fabricate the needle 36 from a material having sufficient strength to pull the skin when the needle 36 is protruded out of the body through the skin and pulled on. The material of the needle 36 obviously must not be bent or broken inside the body during the pulling procedure.

While the above descriptions were provided using as an example a laparoscope inserted into the body of a patient, the laparoscope according to the present embodiment is not necessarily limited to inserting into the body for surgery. It is obvious that the laparoscope may be used for various other applications that include photographing the inside of an object that is difficult to observe with the naked eye, such as for observing a narrow space or the inside of a sealed container.

FIG. 8 is a flowchart illustrating a method of setting a laparoscope according to a preferred embodiment of the present invention. This embodiment relates to a setting process for inserting the housing 10 illustrated in FIG. 5 into the measurement site and obtaining the space required for laparoscopic photography and surgery. In the descriptions that follow, the housing will be referred to as the “laparoscope module.”

As described above, the laparoscope module according to the present embodiment is structured such that the module can be inserted in the body completely without making an incision in the patient's skin for inserting the laparoscope. To set the laparoscope according to the present embodiment, first, the laparoscope module having a hinge-coupled needle may be inserted in the portion being measured (S10).

The needle according to the present embodiment may be hinge-coupled, so that the needle may be folded in the same lengthwise direction as that of the laparoscope module when the laparoscope module is being inserted, and may be unfolded after the laparoscope module is inserted.

After the laparoscope module is inserted in the measurement site, the needle may be rotated such that the needle is orthogonal to the laparoscope module, after which the needle may be protruded to the outside of the measurement site (S20). Outside the measurement site, the protruding needle may be pulled, to provide a space around the laparoscope module for photography (S30).

After laparoscope module is inserted in the measurement site and the space required for photography is obtained, the laparoscope module may be electrically connected with an external device and activated, to proceed with the laparoscopic photography (S40).

In this way, the space for laparoscopic photography can readily be obtained without injecting a gas in the measurement site, simply by pulling the needle that protrudes outwards.

While the present invention has been described with reference to particular embodiments, it is to be appreciated that various changes and modifications can be made by those skilled in the art without departing from the spirit and scope of the present invention as defined by the appended claims.

1. A laparoscope comprising:
a housing extending a particular length;
a pair of lenses set in both end portions of the housing along a lengthwise direction;
a pair of first reflectors installed within the housing adjacent to the pair of lenses, the first reflectors configured to reflect light from the pair of lenses towards a particular position;
a second reflector installed within the housing, the second reflector configured to receive the light reflected from the pair of first reflectors and reflect the light in a particular direction; and
an optical channel coupled to the housing, the optical channel configured to receive the light reflected from the second reflector and transmit the light to a particular position.

2. (canceled)

3. (canceled)

4. (canceled)

5. (canceled)

6. The laparoscope of claim 1, wherein the optical channel is detachably coupled to the housing and a magnet is attached to any one or more of the optical channel and the housing, such that the optical channel is coupled to the housing by magnetic force.

7. The laparoscope of claim 1, wherein the optical channel is detachably coupled to the housing and the optical channel has an alignment protrusion formed on an end portion thereof coupled to the housing, and
the housing has an indentation formed in a portion thereof coupled to the optical channel, the alignment protrusion configured to mate with the indentation.

8. The laparoscope of claim 1, wherein the optical channel is coupled to the housing along a same lengthwise direction as that of the housing.

9. The laparoscope of claim 1, wherein the housing is hinge-coupled to the optical channel, such that the housing is rotatable between an orientation having a same lengthwise direction as that of the optical channel and an orientation orthogonal to the optical channel

10. A laparoscope comprising:
a housing extending a particular length;
a pair of lenses set in both end portions of the housing along a lengthwise direction;
an optical receiver installed within the housing adjacent to the pair of lenses, the optical receiver configured to receive light from the pair of lenses respectively and convert the light into electrical signals; and
an electrical contact electrically connected to the optical receiver and exposed at an exterior of the housing.

11. The laparoscope claim 10, wherein the housing has a cylindrical shape, the housing having a sufficiently large diameter to enable a trocar inserted in a surgical site to pass through.

12. The laparoscope claim 10, wherein the pair of lenses are set in the housing facing a same direction.

13. The laparoscope claim 10, further comprising:
a lighting part coupled to the housing, the lighting part configured to irradiate light towards a direction in which the pair of lenses are facing.

14. The laparoscope of claim 10, further comprising:
a support detachably coupled to the housing, the support extending along a particular lengthwise direction.

15. The laparoscope of claim 14, wherein the support is coupled to the housing along a same lengthwise direction as the housing.

16. The laparoscope of claim 14, wherein the housing is hinge-coupled to the support, such that the housing is rotatable between an orientation having a same lengthwise direction as that of the support and an orientation orthogonal to the support.

17. The laparoscope of claim 10, further comprising:
a needle coupled to the housing.

18. The laparoscope of claim 17, wherein the needle is shaped as a hollow tube, the electrical contact exposed to the exterior through the needle.

19. The laparoscope of claim 17, wherein the electrical contact is formed at an end portion of the needle.

20. The laparoscope of claim 17, wherein the needle is hinge-coupled to the housing, such that the needle is rotatable between an orientation having a same lengthwise direction as that of the housing and an orientation orthogonal to the housing.

21. A method of setting a laparoscope, the method comprising:
inserting a laparoscope module into a measurement site, the laparoscope module having a needle hinge-coupled thereto;
rotating the needle and protruding the needle to an outside of the measurement site;
providing a measurement space around the laparoscope module by pulling the needle; and
activating the laparoscope.

22. The laparoscope of claim 1, wherein the housing has a cylindrical shape, the housing having a sufficiently large diameter to enable a trocar inserted in a surgical site to pass through.

23. The laparoscope of claim 1, wherein the pair of lenses are set in the housing facing a same direction.

24. The laparoscope of claim 1, further comprising:
a lighting part coupled to the housing, the lighting part configured to irradiate light towards a direction in which the pair of lenses are facing.
